(19) United States
(12) Patent Application Publication
(10) Pub. No.: US 2018/0291460 A1
(43) Pub. Date: Oct. 11, 2018

(54) Homologus Recombination Deficiency-Interstitial Aberration (HRD-IA) Assay

(71) Applicant: MAYO FOUNDATION FOR MEDICAL EDUCATION AND RESEARCH, Rochester, MN (US)

(72) Inventors: Ramesh K. RAMANATHAN, Paradise Valley, AZ (US); Mitesh J. BORAD, Tempe, AZ (US); Michael T. BARRETT, Scottsdale, AZ (US)

(21) Appl. No.: 15/766,263

(22) PCT Filed: Oct. 5, 2016
(86) PCT No.: PCT/US16/55466

§ 371 Date: Apr. 5, 2018

Related U.S. Application Data
(60) Provisional application No. 62/238,256, filed on Oct. 7, 2015.

(51) Int. Cl. C12Q 1/6886

(52) U.S. Cl. CPC C12Q 1/6886; C12Q 2600/112; C12Q 2600/118; C12Q 2600/156; C12Q 2600/106

(57) ABSTRACT

Methods for sorting out cancer sub-types based on sensitivity to DNA damaging drugs or inhibitors of DNA repair are described so that patients can be selected as candidates for treatment with these agents.

### FIELD OF THE INVENTION

Methods for the identification of cancer sub-types based on sensitivity to DNA damaging drugs or inhibitors of DNA repair are described so that patients can be selected as candidates for treatment with these agents.

### BACKGROUND OF THE INVENTION

Success in treating particular cancers is hampered by the fact that the cancer is often highly evolved by the time it is diagnosed, heterogeneous and resistant to standard drug treatment. Pancreatic cancer, colon cancer and breast cancer are good examples of these problems.

Pancreatic cancer is diagnosed in more than 40,000 people in the U.S. each year, with the vast majority dying from the disease. In Europe the numbers are even higher, with over 60,000 diagnosed each year. Surgery is usually not practical for the majority of cases. Radiation is a contested therapy, with some researchers indicating that radiation stimulates the growth, invasion and metastases of pancreatic cancer. Chemotherapeutics, even in combination, provide only modest (weeks to months) improvements in survival. Overall, median survival from diagnosis is around 3 to 6 months; 5-year survival is less than 6 percent.

In sheer numbers, colon cancer is even a bigger killer. With over 650,000 deaths worldwide per year, it is the third most common form of cancer and the second leading cause of cancer-related death in the Western world. When detected late, surgery may be of no use. For example, 20% of patients present with metastatic (stage 1V) colorectal cancer at the time of diagnosis, and only 25% of this group will have an isolated liver metastasis that is potentially resectable. Radiation is not routinely used since it can cause radiation enteritis. Chemotherapy is often used post-surgery as adjunct therapy.

Breast cancer is the most common malignancy and the second leading cause of cancer death in women. In over 60% of localized breast cancer cases, histological evidence of tumor spread to surrounding tissue is found. Patients diagnosed with invasive ductal carcinoma, the most common breast cancer, have a lower 10-year survival rate. About 30% of newly diagnosed breast cancer patients have positive lymph nodes and much poorer outcomes.

What is needed are better ways to profile and classify cancers so that patients can be matched up with more appropriate drugs for treatment.

### SUMMARY OF THE INVENTION

Methods for the identification of cancer sub-types based on sensitivity to DNA damaging drugs or inhibitors of DNA repair are described so that patients can be selected as candidates for treatment with these agents.

In one embodiment, the present invention contemplates a method of profiling and classifying solid tumors for interstitial chromosomal aberrations, comprising, a) providing a sample of a solid tumor from a patient, and b) identifying ex vivo a number of interstitial chromosomal aberrations in the genome of said solid tumors, wherein said number is above a threshold number. In a preferred embodiment, the interstitial chromosomal aberrations (IAs) are aberrant copy number intervals with sub chromosomal boundaries. In a preferred embodiment, aberrations in chromosome number (gain of chromosomes or a deficiency in chromosomes) are not identified as interstitial chromosomal aberrations (IAs). In a preferred embodiment, copy number neutral aberrations including inversions, balanced translocations and ring chromosomes are not identified as interstitial chromosome aberrations (IAs). The threshold number distinguishes HRD-positive from HRD-negative genomes. In one embodiment, said threshold number is established by the number of interstitial chromosomal aberrations observed in homologous recombination deficient (HRD)-positive BRCA^{mut} tumors, i.e. the threshold number is 50 or more, and more preferably greater than 50 interstitial chromosomal aberrations (IAs), or the threshold number is 40 or more, and more preferably greater than 40 IAs, or the threshold number is 30 or more, and more preferably greater than 30 IAs.

In one embodiment, the solid tumor tests negative for a BRCA mutation; said another way, said solid tumor tests as wild type for BRCA (BRCA^{wt}). In a preferred embodiment, the mutation status of any particular gene is not tested or identified.

In one embodiment, the present invention contemplates that an elevated number of IAs is diagnostic for, and permits the selection of, those patients with solid tumors who will respond to treatment with DNA damage and repair targeting agents, regardless of the mutation status of any particular gene (i.e. whether BRCA^{wt} or BRCA^{wt}). Thus, in one embodiment, the method further comprises c) treating said patient having said solid tumor identified to have interstitial aberrations above the threshold in step b) with one or more DNA damaging agents, one or more DNA repair targeting agents (e.g. inhibitors of DNA repair), or a combination thereof.

In one embodiment, we have combined array based comparative genomic hybridization (aCGH) assays with flow sorted samples to identify solid tumors, and in particular PDAs, with copy number changes indicating sensitivity to nucleic acid damaging agents and/or nucleic acid repair inhibitors.

In one embodiment, the present invention contemplates a method of testing (and profiling and classifying) solid tumors for interstitial chromosomal aberrations comprising a) providing a sample of a solid tumor from a patient; b) isolating nucleic acid from said sample; c) treating said nucleic acid under conditions such that the total number of interstitial chromosomal aberrations in the genome of said solid tumor is identified, said interstitial chromosomal aberrations consisting of aberrant copy number intervals with sub-chromosomal boundaries; and d) notifying said patient's treating physician that said patient is a candidate for nucleic acid damaging agents or repair inhibitors, wherein said total number of interstitial chromosomal aberrations is above 50. A variety of sources of tumor samples are contemplated. In one embodiment said providing step comprises obtaining a biopsy. In one embodiment, the present invention contemplates purifying or isolating tumor cells from the sample so that the tumor cells are 95% pure or greater. For example, in one embodiment, the method further comprises prior to step b), isolating tumor cells from said solid tumor such that said tumor cells are free of non-tumor cells (or substantially free, e.g. less than 5% non-tumor cells, or less than 3% non-tumor cells, or less than 1% non-tumor cells, or less than 0.1% non-tumor cells). The entire tumor cell need not be utilized. For example, in one embodiment, the present invention contemplates prior to step b) isolating nuclei of the tumor cells from said solid tumors. In one embodiment, the method further comprises, prior to step b) separating diploid nuclei from non-diploid nuclei. Flow sorting can be used to isolate tumor cells, or nuclei; it can also be used to separate diploid nuclei from non-diploid. In one embodiment, the present invention contemplates said isolating comprises single parameter or multi-parameter (two parameters, three parameters, etc.) flow sorting. In one embodiment, said treating of step c) comprises exposing said nucleic acid to a copy number array. In one embodiment, the method further comprises e) treating said solid tumor of said patient with at least one nucleic acid damaging agent. A variety of DNA damaging agents are contemplated. In one embodiment, said at least one nucleic acid damaging agent is an alkylating agent. In one embodiment, said alkylating agent is a metal salt. In one embodiment, said metal salt is selected from the group consisting of Carboplatin, Cisplatin, and Oxaliplatin. Treatment can also extend to the use of other drugs, whether alone or in combination. For example, in one embodiment, the method further comprises e) treating said solid tumor of said patient with at least one nucleic acid repair inhibitor. A variety of repair inhibitors are contemplated. In one embodiment, said at least one nucleic acid repair inhibitor is a polymerase inhibitor. In one embodiment, said polymerase inhibitor is an inhibitor of poly ADP ribose polymerase (PARP). In one embodiment, said inhibitor is Olaparib. The present invention is useful generally with solid tumors. In one embodiment, said solid tumor is a pancreatic tumor. In one embodiment, said solid tumor is pancreatic ductal adenocarcinoma (PDA). In one embodiment, said solid tumor is a cancer of the brain, ovary, breast, colon, or other solid tissue tumors. In one embodiment, the method further comprises e) treating said solid tumor of said patient with a polychemotherapeutic (i.e. multiple drug) regimen. Examples of a “polychemotherapeutic regimen” multiple drug regimen include but are not limited to FOLFOX, a combination of FOL—Folinic acid (leucovorin), F—Fluorouracil (5-FU), OX—Oxaliplatin (Eloxatin); FOLFIRINOX, a combination of fluorouracil [5-FU], leucovorin, irinotecan and oxaliplatin; a modified FOLFIRINOX, including Onivyde, 5-FU, a liposomal form of leucovorin; a modified FOLFIRINOX+Pegylated Recombinant Human Hyaluronidase (PEGPH20); NAPLAGEM, a combination of nab-paclitaxel+oxaliplatin+gemcitabine; a combination of evofosfamide/nab-paclitaxel/Gemcitabine; a combination of Evofosfamide and Gemcitabine; etc. Evofosfamide (TH-302) refers to a hypoxia-activated pro-drug of bromo-isophosphoramide mustard (Br-IPM).

In one embodiment, the present invention contemplates a method of treating patients having solid tumors comprising a) providing a sample of a solid tumor from a patient, b) isolating nucleic acid from said sample, and c) subjecting at least a portion of said nucleic acid to conditions such that the total number of interstitial chromosomal aberrations in the genome of said solid tumor is identified, said interstitial chromosomal aberrations consisting of aberrant copy number intervals with sub-chromosomal boundaries, and d) treating said patient having said solid tumor, when said total number is above 50, with at least one nucleic acid damaging agent or at least one nucleic acid repair inhibitor or both. In one embodiment, said patient was previously treated with a chemotherapeutic drug to which said solid tumor is resistant. A variety of sample sources are contemplated. In one embodiment, said providing step comprises obtaining a biopsy. It is useful to isolate or purify tumor cells or portions thereof. In one embodiment, the method further comprises, prior to step b), isolating tumor cells from said solid tumor such that said tumor cells are free of non-tumor cells. In one embodiment, the method further comprises, prior to step b) isolating nuclei of the tumor cells from said solid tumor. In one embodiment, the method further comprises, prior to step b) separating tumor nuclei from non-tumor nuclei. In one embodiment, said isolating comprises single parameter or muliparamter flow sorting. In one embodiment, said isolating comprises DNA content-based flow sorting. In one embodiment, said subjecting to conditions of step c) comprises exposing said nucleic acid to a copy number array. A variety of damaging agents is contemplated. In one embodiment, said at least one nucleic acid damaging agent is an alkylating agent. In one embodiment, said alkylating agent is a metal salt. In one embodiment, said metal salt is selected from the group consisting of Carboplatin, Cisplatin, and Oxaliplatin. Repair inhibitors can also be utilized in such selected patients. In one embodiment, said at least one nucleic acid repair inhibitor is a polymerase inhibitor. In one embodiment, said polymerase inhibitor is an inhibitor of poly ADP ribose polymerase (PARP). In one embodiment, said inhibitor is Olaparib. Again, the approach is useful generally for solid tumors. In one embodiment, said solid tumor is a pancreatic tumor. In one embodiment, said solid tumor is pancreatic ductal adenocarcinoma (PDA). In one embodiment, said solid tumor is a cancer of the brain, ovary, breast, colon, or other solid tissue tumors.

The present invention contemplates combining features from different embodiments. For a non-limiting example, the use of the inventive assay to determine interstitial chromosomal aberrations (or interstitial aberrations) as described herein may be used for in combination with other medical diagnostic tests. As another example, individual drugs of combinations may be used to make new combinations of chemotherapeutics. The present invention contemplates removing features from the above-indicated embodiments. For a non-limiting example, irinotecan and oxaliplatin may be used instead of the entire FOLFIRINOX combination. As another example, breast cancer cells described, might not be triple negative, for example, they may be PR and/or ER negative but not HER2 negative, i.e. the cells may be PR and ER negative but not over-express HER2 (such that low levels of HER2 are detectable), or the cancer cells may be PR negative or ER negative while over-expressing HER2. The present invention contemplates substituting features in the above-indicated embodiments. For a non-limiting example, embodiments specifically describing DNA damaging agents may have substitutions of other types of DNA damaging agents. As another example, embodiments specifically describing repair inhibitors may have substitutions of other types of repair inhibitors. As another example, embodiments specifically describing chemotherapeutic drug combinations may have substitutions of other drugs for one or more drugs in the combinations.

### Definitions

Solid tumors include, but are not limited to, pancreatic cancer, colon cancer and breast cancer, glioblastomas, bladder carcinoma, and small cell carcinoma of the ovary.

Nucleic acid damaging agents or DNA damaging agents are agents that modify or damage nuclei acid. In one embodiment, such damaging agents are alkylating agents. There are several types of alkylating agents including 1) Mustard gas derivatives: Mechlorethamine, Cyclophosphamide, Chlorambucil, Melphalan, and Ifosfamide; 2) Ethylenimines: Thiotepa and Hexamethyl-melamine; 3) Alkylsulfonates: Busulfan; 4) Hydrazines and Triazines: Procarbazine, Dacarbazine and Temozolomide; 5) Nitrosureas: Carmustine, Lomustine and Streptozocin; and 6) Metal salts: Carboplatin, Cisplatin, and Oxaliplatin. Nitrosureas are unique because, unlike most chemotherapy, they can cross the blood-brain barrier. They can be useful in treating brain tumors.

Nucleic acid repair inhibitors are agents, which inhibit the function of components of a repair pathway, such as an inhibitor of poly ADP ribose polymerase (PARP). While not intending to be limited to any one particular inhibitor, Olaparib (AZD-2281, trade name Lynparza) is an FDA-approved inhibitor. Poly(ADP-ribose) polymerases (PARP) comprise a family of at least eighteen proteins containing PARP catalytic domains (Amé et al. BioEssays (2004) 26:882, herein incorporated by reference). These proteins include PARP-1, PARP-2, PARP-3, tankyrase-1, tankyrase-2, and others. PARP inhibitors interact with the nicotinamide binding domain of the enzyme and behave as competitive inhibitors with respect to NAD+ (Ferraris, J. Med. Chem. (2010) 53(12):4561-4584 and Bundschere et al, Anti-Cancer Agents in Medicinal Chemistry (2009) 9:816-821, each of which are herein incorporated by reference). Thus, structural analogues of nicotinamide, such as benzamide and derivatives are examples of PARP inhibitors. Amide or aryl substituted 4-benzyl-2H-phthalazin-1-ones derivatives were disclosed as inhibitors of PARP, e.g. in WO 2002/036576, WO 2003/070707, WO 2003/093261, WO 2004/080976, WO 2007/045877, WO 2007/138351, WO 2008/114023, WO 2008/122810, and WO 2009/093032, each of which are herein incorporated by reference. Certain amide substituted 6-benzylpyridazin-3(2H)-one derivatives were disclosed as potent inhibitors of PARP enzymes, e.g. in WO 2007/138351, US20080161280, US2008/0269234, WO2009/004356, WO2009/063244, and WO 2009/034326, each of which are herein incorporated by reference. Additionally, PARP inhibitors are described in WO 2012166983, herein incorporated by reference. Specific non-limiting examples include rucaparib (CO-338; AG014699, PF-0367338; oral/IV), iniparib (BSI-201), olaparib (AZD-2281; oral), veliparib (ABT-888; oral), MK-4827, BMN-673, CEP-9722 (oral) and E7016 (GPI 21016, oral).

### BRIEF DESCRIPTION OF THE DRAWINGS

FIG. 1 shows DNA content based sorting of solid tumors. FIG. 1A) is a schematic of a work flow where single particle suspensions of nuclei are prepared from biopsies of tumors of interest. FIG. 1B) is a Histogram of DAPI (4′,6-diamidino-2-phenylindole) stained nuclei which identifies 4 distinct populations within a single biopsy from a PDA surgical resection. FIG. 1C) is a schematic showing gating on each population allows simultaneous collection of each of the 4 populations in separate tubes for downstream genomic analyses.

FIG. 2 shows an analysis of a BRCA2^{mut} PDA genome. A diploid (2.0N) and an aneuploid (3.9N) population were sorted from a needle biopsy from a liver metastasis (upper left panel). The 3.9N population had multiple genomic aberrations throughout the genome while the 2.0N was normal by aCGH (bottom panels). Over 50 IAs were detected in the 3.9N genome including deletions at 9p23-p13.2 and 13q21.31-q33.3 (upper right panels). In each case an additional homozygous deletion (blue arrows) targeting PDA associated tumor suppressor genes (CDKN2A, SLITRK5, SLITRK6) was internal to the hemizygous deletion. Shaded red areas denote ADM2 step gram defined IA.

FIG. 3 is a bar graph showing interstitial copy number aberrations in Stand up to Cancer (SU2C) trial 2026001. Liver metastases from patients with metastatic PDA who progressed on prior therapies were profiled by flow cytometry and aCGH. The patients were then ranked according to the number of interstitial aberrations detected in the tumor genomes.

FIG. 4 shows a comparison of BRCA2^{wt} SU2C-46 genome with BRCA2^{mut} genomes of breast (PS13-1750), PDA (PDA-B01), and ovarian (OvCa 17) tumors. Each genome had >50 IAs based on aCGH with sorted tumor nuclei.

FIG. 5 shows a comparison of matching sorted FFPE and FF PDA samples. Flow sorting histogram of 3.2N tumor population in matched FF tissue and FFPE tissue (left panels). Gene view comparison of copy number aberrations. Chromosome 9p22.2 region includes a homozygous deletion of CDKN2A (black arrow) and a focal amplicon of SH3GL2 (blue arrow). Chromosome 2p14 region includes a focal amplicon with the MEIS1 gene (red arrow). Shaded areas denote ADM2 copy number aberrant intervals.

FIG. 6 shows an example of a HRD-IA assay-using patient TNBC-1 (i.e. MET694) samples. An exemplary result is shown for TNBC-1's result for human Chromosome 17 in the area of 17q23.2 using BRCA2^{wt} triple negative breast cancer cells (TNBCs). The results show a homozygous deletion of BRIP1 (a regulator of BRCA).

FIG. 7 shows an example of comparative HRD-IA assay-using patient TNBC-2 (i.e. PAD758) samples. An exemplary result is shown for TNBC-2>50 between tumor tissue and normal tissue on human Chromosome 10 in the region of the DCLRE1C (DNA Cross-Link Repair 1C) gene encoding an Artemis protein. The BRCA2^{wt} triple negative breast cancer tumor tissue shows a 16 bp deletion in the DCLRE1C gene.

### DETAILED DESCRIPTION OF THE INVENTION

Methods for the identification of cancer sub-types based on sensitivity to DNA damaging drugs or inhibitors of DNA repair are described so that patients can be selected as candidates for treatment with these agents.

Currently, there are a series of homologous recombination deficiency HRD genotype assays in development for clinical application. These include the HRD loss of heterozygosity (HRD-LOH) score, the HRD large-scale transition (HRD-LST) score, the HRD telomeric allelic imbalance (HRD-TAI), and a score combining breakpoints dispersed over large regions with tumor ploidy. See Popova, T., et al., Ploidy and large-scale genomic instability consistently identify basal-like breast carcinomas with BRCA1/2 inactivation. Cancer Research, 72(21): p. 5454-62 (2012); Abkevich, V., et al., Patterns of genomic loss of heterozygosity predict homologous recombination repair defects in epithelial ovarian cancer. British J. Cancer, 107(10): p. 1776-82 (2012); Birkbak, N.J., et al., Telomeric allelic imbalance indicates defective DNA repair and sensitivity to DNA-damaging agents. Cancer Discovery, 2(4): p. 366-75 (2012); Wang, Z. C., et al., Profiles of genomic instability in high-grade serous ovarian cancer predict treatment outcome. Clinical Cancer Res. 18(20): p. 5806-15 (2012), each of which are herein incorporated by reference. These approaches, which can be combined for composite scores, typically rely on short oligonucleotide based single nucleotide polymorphism (SNP) platforms for detection of a BRCA^{mut} genomic signature. To advance these assays for clinical use, biopsies are typically assessed for sufficient tumor content by conventional histopathology review prior to analysis and data are normalized accordingly using algorithms of choice.

Although published estimates propose that SNP array and sequencing based HRD assays can be used in the presence of >30% admixtures, these claims have not been rigorously validated in the clinic. For example initial studies of the performance of SNP based platforms described high sensitivity to normal cell admixtures in the detection of copy number changes and the discrimination of homozygous deletions from partial deletions in simple mixing experiments Zhao, X., et al., An integrated view of copy number and allelic alterations in the cancer genome using single nucleotide polymorphism arrays. Cancer Res, 64(9): p. 3060-71 (2004), herein incorporated by reference. Short SNP oligonucleotide probes (i.e. ≤30 nucleotides) have low resolution on a probe by probe basis for detection of copy number changes using genomic DNA targets. Furthermore SNP based assays require patient matched normal samples as controls for accurate clinical tests.

In contrast, 50-60mer oligonucleotide probes can be designed for high resolution copy number measurements with total genomic DNA targets and universal references (Barrett et al., PNAS, 101(51): p. 17765-17770 (2004), herein incorporated by reference. In one embodiment, the present invention contemplates using such probes in a copy number array for measuring copy number changes in solid tumors. In one embodiment the copy number arrays are Agilent Sure Select arrays with 180,000, 244,000, 400,000, or 1,000,000 60-mer probes (available commercially). In one embodiment the arrays are custom CGH arrays with 60-mer probes designed to cover the entire genome and genes and regions of interest. In one embodiment, the present invention contemplates a combination of flow sorted clinical samples (in order to isolate tumor cells from non-tumor cells, matrix and debris) and high resolution copy number assays (e.g. aCGH assays) in order to provide a HRD-IA score that can be immediately applied to identify sub-types of PDA and other solid tumors that will be sensitive to nucleic acid damaging agents and/or nucleic acid repair inhibitors.

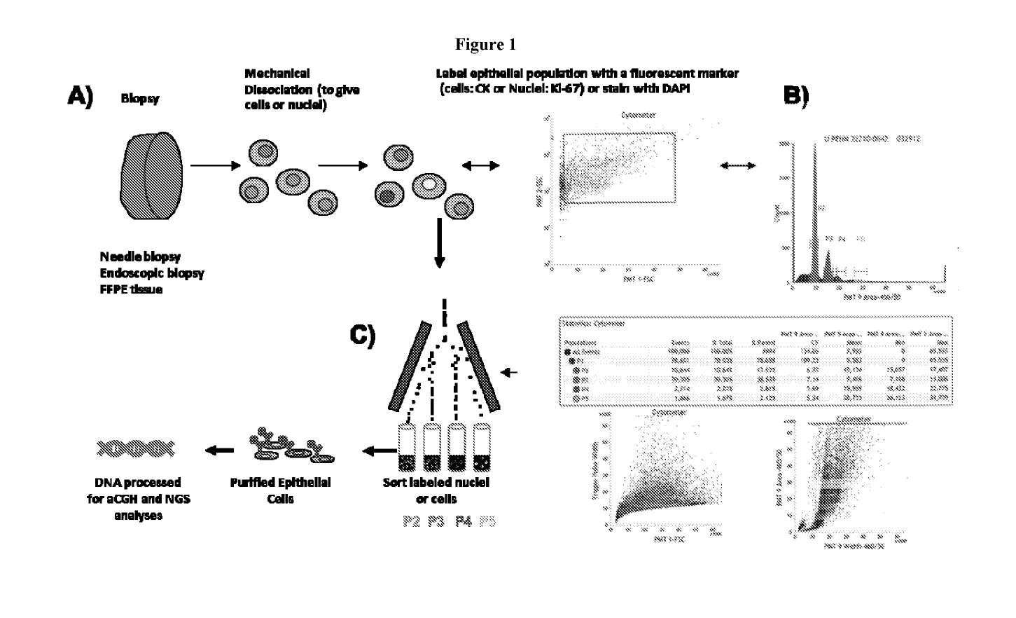

Solid tumors are difficult to molecularly characterize at the biopsy level due to complex genomes and heterogeneous cellularity, as cancer cells may represent a small fraction of the cells within the tumor. Furthermore, clinical samples frequently contain multiple neoplastic populations that cannot be distinguished by morphology based methods. In one embodiment, the present invention contemplates addressing these problems with a combination of flow sorting and aCGH based HRD-IA assay as a robust method to identify patients whose tumors will respond to DNA damage and repair targeting agents. In one embodiment, flow sorting of cells or nuclei from a tumor biopsy (FIG. 1) is used to identify distinct diploid, tetraploid and aneuploid tumor populations in the solid tumor (such as PDA). In one embodiment, flow sorting of cells or nuclei combines DNA measures with markers for tumor properties including proliferation, differentiation, and activated cellular signaling pathways. In one embodiment, highly purified (>95% tumor cells) samples are obtained prior to whole genome analyses. While fresh biopsied material is preferred, a variety of clinical samples can be treated in this manner, including both fresh frozen and formalin fixed paraffin embedded (FFPE) tissues with low tumor cell content (<10-20%) and high amounts (>90%) of necrosis and debris.

We have identified PDAs and other solid tumors (e.g. breast, colon, etc.) with extensive numbers of interstitial aberrations (IAs) in their genomes similar to those observed in HRD-positive BRCA^{mut} TNBCs.

Our results support that a HRD score based on elevated numbers of IAs (HRD-IA) correlates with clinical response of PDA and other solid tumors to DNA damage targeting agents. This score only considers copy number variations and excludes other chromosomal aberrations, making it simple and robust. We propose that the combination of single or multiparameter DNA content flow sorting and array based comparative genomic hybridization (aCGH) analyses is a unique opportunity to identify those PDAs and other solid tumors that are responsive to DNA damage targeting agents and repair inhibitors.

In one embodiment, the present approach does not involve any sequencing. While current efforts in next-generation sequencing are targeting high number reads (e.g., >100×) to overcome tissue heterogeneity, this is not an optimum approach. Increasing read number will only exacerbate errors associated with poor quality samples.

Purified flow sorted fresh frozen tissue samples can provide inputs for whole genome sequencing analysis including HRD scores. Therefore, in one embodiment, the present invention contemplates purifying fresh frozen tumor cells by sorting, followed by whole genome sequencing analysis including HRD scores. In one embodiment, the sequencing is sequencing by synthesis (SBS), wherein specially designed nucleotides and DNA polymerases are used to read the sequence of immobilized, single-stranded DNA templates in a controlled manner. See U.S. Pat. Nos. 6,664,079 and 8,481,259, hereby incorporated by reference. In particular, the protocols for SBS are hereby incorporated by reference. However next generation sequencing (NGS) of FFPE tumor samples are limited to targeted approaches from candidate genes to whole exome analysis (Holley et al 2012 PLosOne). NGS results for FFPE tissues do not provide whole genome based HRD analysis (Telli et al., Clin Canc Res 2016). In addition HRD assays that incorporate measures of allele heterozygosity require patient matched normal samples. In contrast our aCGH based HRD-IA assay provides whole genome coverage without the need for patient matched samples with fresh frozen and FFPE tissue samples.

### EXPERIMENTAL

### Example 1

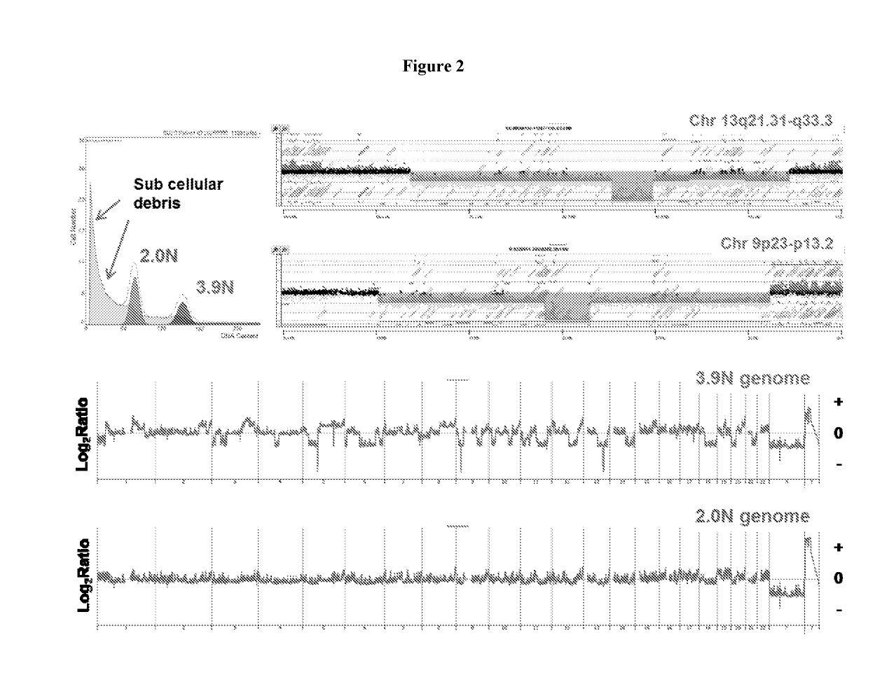

The clinical significance of our HRD-IA assay is highlighted in the analyses of a needle biopsy from a liver metastasis obtained in our recently completed Stand up to Cancer (SU2C) sponsored clinical trial of patients with PDA who progressed on prior therapies (FIG. 2). We sorted a diploid (2.0N) and an aneuploid (3.9N) population from the biopsy then profiled their genomes with aCGH. Notably there was a high level of subcellular debris detected in the sorting histogram (FIG. 2, upper left panel). This level of debris is frequently seen in heavily pre-treated tumors. The aneuploid genome had over 50 IAs that included 20 of 22 autosomes. Each IA was defined by the ADM2 step gram algorithm as a copy number aberrant interval with intrachromosomal boundaries. Barrett, et al., Comparative genomic hybridization using oligonucleotide microarrays and total genomic DNA. Proc Natl Acad Sci USA, 2004. 101(51): p. 17765-70; Lipson, et al., Efficient calculation of interval scores for DNA copy number data analysis. J Comput Biol, 13(2): p. 215-28 (2006), each of which are herein incorporated by reference.

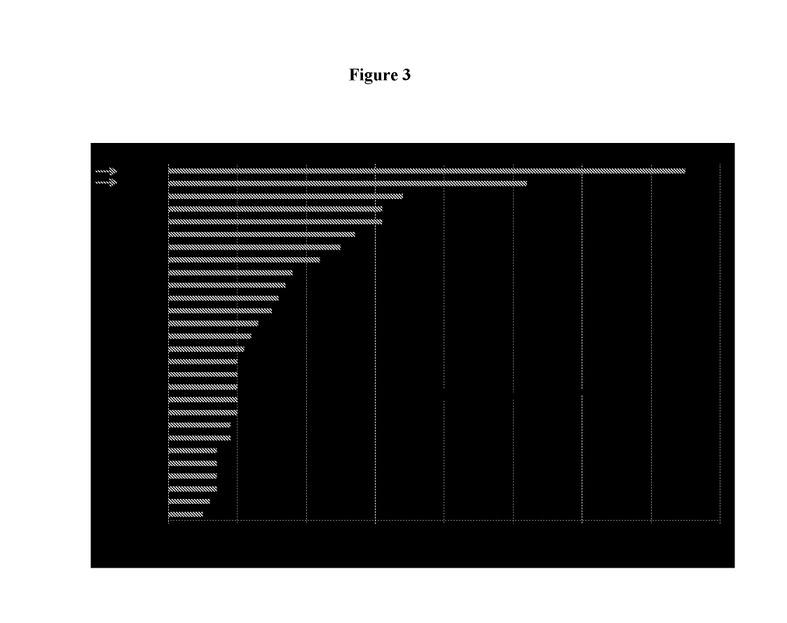

These IAs included deletions and homozygous losses in the tumor genome (but not the deletion or loss of an entire chromosome). In contrast the diploid genome was copy number neutral. This patient (SU2C-6) was verified in a CLIA setting to be a BRCA2^{mut} carrier. A summary of the patients in the trial showed a range of <10 to >70 in the number of interstitial aberrations in each sorted tumor population (FIG. 3). Notably the highest number of aberrations was observed in the sorted aneuploid population from PDA patient (SU2C-46). The latter had a stable disease response to a PARP inhibitor prior to enrollment in the SU2C trial. Strikingly, this patient was wild type for BRCA1 and BRCA2 based on CLIA assays.

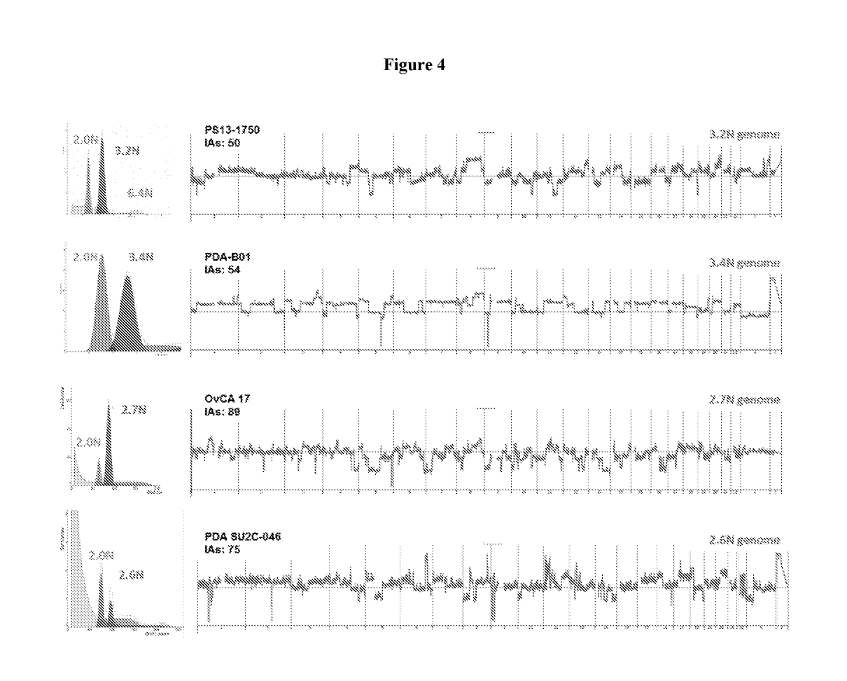

A comparison of SU2C-46 with BRCA^{mut} tumors we have profiled further highlights the potential clinical significance of our HRD-IA scoring assay (FIG. 4). These included breast, ovarian, and PDA tumors with research biopsies obtained from protocols that included primary tumor at time of surgery (breast PS13-1750), metastases during clinical trials (PDA-B01), and resections from tissue archives (OvCa-17). In all cases samples were sorted prior to aCGH analysis, enabling high-resolution detection of IAs (n=50-89) in each genome regardless of tumor content and the levels of subcellular debris.

Further, we discovered that one of the cases shown in FIG. 4 (PDA-B01; second chart down from the top) has a BRCA2 variant of unknown significance (a VUS, i.e. a variant of uncertain significance). VUS is a common event associated with BRCA1 and BRCA2 as well as other DNA repair genes. This patient did have a very strong response to FOLFOX. FOLFOX refers to a chemotherapy regimen for treatment of colorectal cancer, made up of the drugs FOL—Folinic acid (leucovorin), F—Fluorouracil (5-FU) and OX—Oxaliplatin (Eloxatin).

These results highlight that our assay is a phenotype based assay and can provide novel information about each patient.

### Example 2

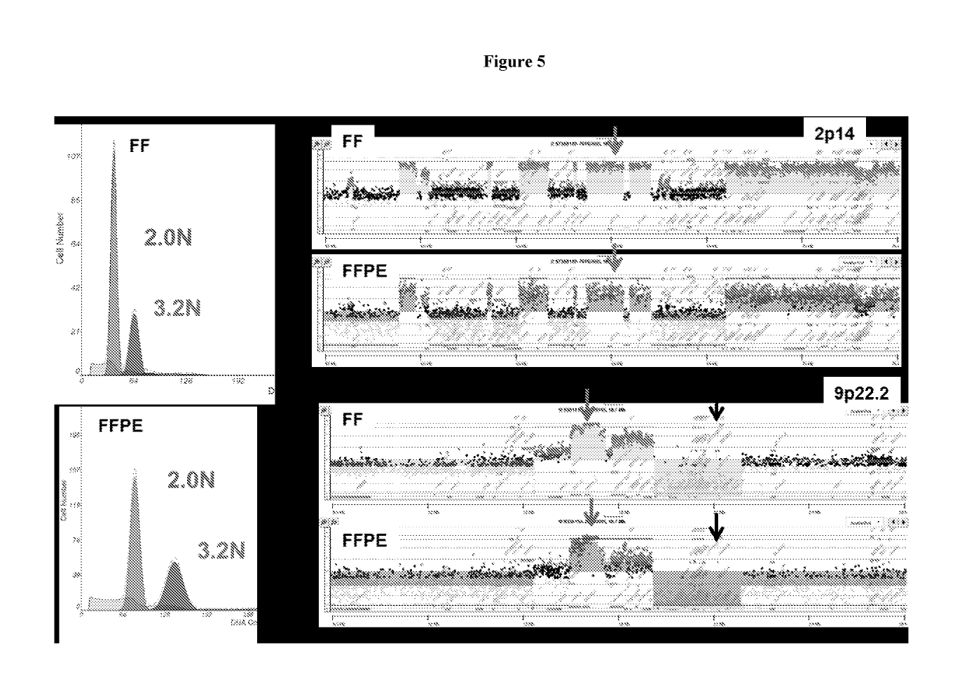

In this example, we evaluated the use of sorted solid tissue FFPE samples by selecting PDA samples with matching FF material. In each case, we sorted a minimum of 50,000 aneuploid and diploid nuclei from the FFPE samples and a minimum of 10,000 nuclei from the same populations in the matching FF samples. The width of the histograms for the diploid and aneuploid (3.2N) peaks in a liver metastasis was greater for the FFPE sample likely reflecting the lower quality of the sample relative to the FF sample (FIG. 5). DNA from the sorted FF sample was prepared by our methods. After hybridization and feature extraction we used the ADM2 intervals to measure the reproducibility of aCGH data in the matching FFPE and FF samples. Two intervals were called similar if their genomic regions overlapped by more than 0.5. The overlap of two intervals is defined as the genomic length of their intersection divided by the genomic length of their union. We selected the top 20 ranked amplicons in the FFPE sample for this analysis. In 19 of these 20 amplicons the overlap was >0.9 with the same ADM2-defined interval in the sorted FF sample. These intervals included a series of focal amplicons on chromosomes 9 and 2 that targeted known (SH3GL2) and putative (MEIS1) oncogenes and a homozygous deletion of the tumor suppressor CDKN2A. We subsequently extended this approach to FFPE samples from a variety of solid tumor tissues, including triple negative breast cancers (TNBCs), glioblastomas, bladder carcinoma, and small cell carcinoma of the ovary, to validate our methods. Our ability to sort and profile FFPE tissues extends the application of our HRD-IA assay to a wide variety of clinical samples.

### Example 3

This example shows additional results of HRD-IA assays on homologous recombination deficient (HRD)-positive triple negative breast cancers (TNBC) that were BRCA wild type. Triple negative breast cancer refers to a cancer cell population diagnosed as lacking receptors for estrogen, progesterone and human epidermal growth factor (Her2), denoted ER-, PR-, and HER2-, respectively. In some embodiments, triple negative breast cancer cells have low to 0 levels of detectable receptors for estrogen and/or progesterone, and/or HER2 receptors.

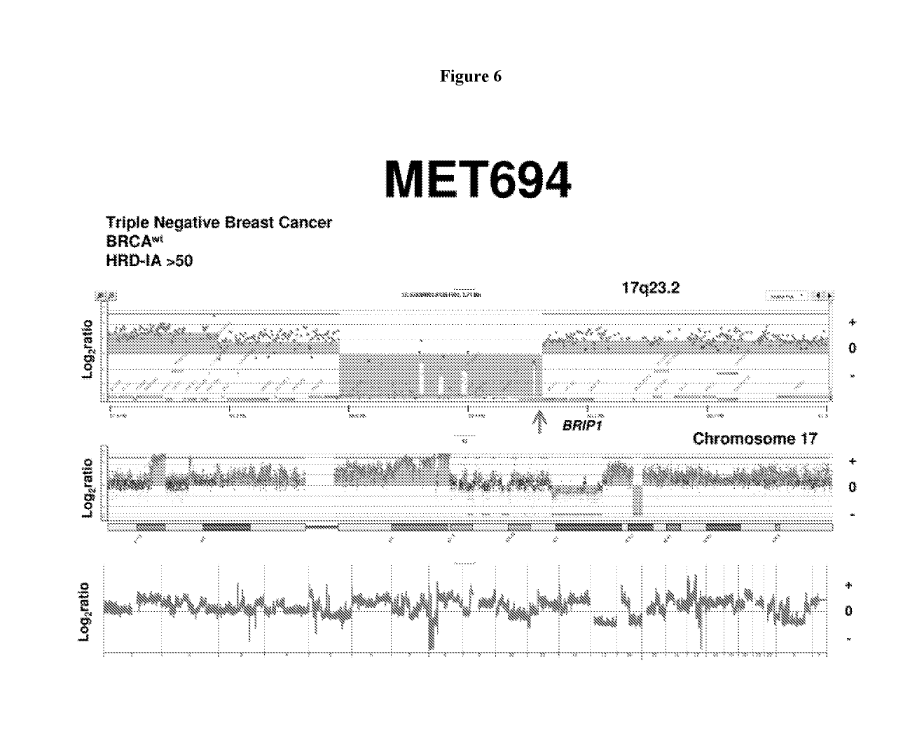

FIG. 6 shows an example of a HRD-IA assay-using patient TNBC-1 (i.e. MET694) samples. An exemplary result is shown for TNBC-1's human Chromosome 17 in the area of 17q23.2 using BRCA2^{wt} triple negative breast cancer cells. These results show a homozygous deletion of BRIP1 (a regulator of BRCA).

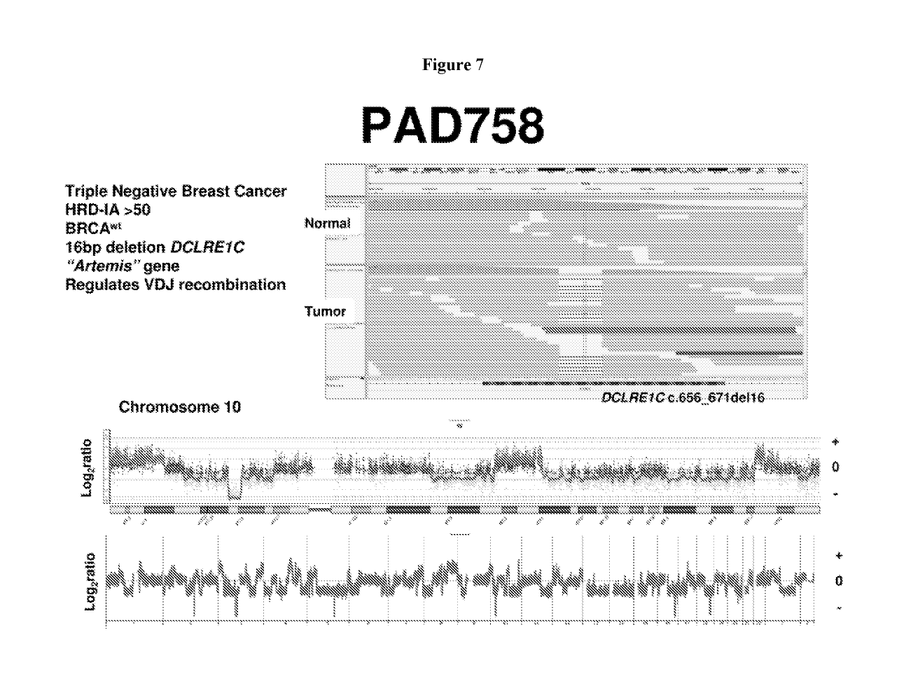

FIG. 7 shows an example of comparative HRD-IA assay-using patient TNBC-2 (i.e. PAD758) samples. An exemplary result is shown for TNBC-2>50 between tumor tissue and normal tissue on human Chromosome 10 in the region of the DCLRE1C (DNA Cross-Link Repair 1C) gene encoding an Artemis protein. The BRCA2^{wt} triple negative breast cancer tumor tissue shows a 16 bp deletion in the DCLRE1C gene. Thus this patient/case has a 16 bp indel in the ARTEMIS gene (a regulator of VDJ recombination). Indel refers to the insertion or the deletion of bases in a gene; in this case/example, there is a deletion.

These results highlight the ability of this HRD-IA assay to identify HRD positive patients and to discover the genetic basis of the phenotype.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in medicine, molecular biology, cell biology, genetics, statistics or related fields are intended to be within the scope of the following claims.

1. A method of testing solid tumors for interstitial chromosomal aberrations, comprising, a) providing a sample of a solid tumor from a patient; b) isolating nucleic acid from said sample; c) treating said nucleic acid under conditions such that the total number of interstitial chromosomal aberrations in the genome of said solid tumor is identified, said interstitial chromosomal aberrations consisting of aberrant copy number intervals with sub-chromosomal boundaries; and d) notifying said patient's treating physician that said patient is a candidate for nucleic acid damaging agents or repair inhibitors, wherein said total number of interstitial chromosomal aberrations is above 50.

2. The method of claim 1, wherein said providing step comprises obtaining a biopsy.

3. The method of claim 1, further comprising, prior to step b), isolating tumor cells from said solid tumor such that said tumor cells are free of non-tumor cells.

4. The method of claim 3, further comprising, prior to step b) isolating nuclei of the tumor cells from said solid tumors.

5. The method of claim 4, further comprising, prior to step b) separating diploid nuclei from non-diploid nuclei.

6. The method of claim 3, wherein said isolating comprises single parameter or multiparameter flow sorting.

7. The method of claim 1, wherein said treating of step c) comprises exposing said nucleic acid to a copy number array.

8. The method of claim 1, further comprising e) treating said solid tumor of said patient with at least one nucleic acid damaging agent.

9. The method of claim 8, wherein said at least one nucleic acid damaging agent is an alkylating agent.

10. The method of claim 9, wherein said alkylating agent is a metal salt.

11. The method of claim 10, wherein said metal salt is selected from the group consisting of Carboplatin, Cisplatin, and Oxaliplatin.

12. The method of claim 1, further comprising e) treating said solid tumor of said patient with at least one nucleic acid repair inhibitor.

13. The method of claim 12, wherein said at least one nucleic acid repair inhibitor is a polymerase inhibitor.

14. The method of claim 13, wherein said polymerase inhibitor is an inhibitor of poly ADP ribose polymerase (PARP).

15. The method of claim 14, wherein said inhibitor is Olaparib.

16. The method of claim 1, wherein said solid tumor is a pancreatic tumor.

17. The method of claim 16, wherein said solid tumor is pancreatic ductal adenocarcinoma (PDA).

18. The method of claim 1, wherein said solid tumor is a cancer of the brain, ovary, breast, or colon.

19. The method of claim 1, further comprising e) treating said solid tumor of said patient with a polychemotherapeutic regimen.

20. A method of treating patients having solid tumors comprising a) providing a sample of a solid tumor from a patient, b) isolating nucleic acid from said sample, and c) subjecting at least a portion of said nucleic acid to conditions such that the total number of interstitial chromosomal aberrations in the genome of said solid tumor is identified, said interstitial chromosomal aberrations consisting of aberrant copy number intervals with sub-chromosomal boundaries, and d) treating said patient having said solid tumor, when said total number is above 50, with at least one nucleic acid damaging agent or at least one nucleic acid repair inhibitor or both.

21. The method of claim 20, wherein said patient was previously treated with a chemotherapeutic drug to which said solid tumor is resistant.

22. The method of claim 20, wherein said providing step comprises obtaining a biopsy.

23. The method of claim 20, further comprising, prior to step b), isolating tumor cells from said solid tumor such that said tumor cells are free of non-tumor cells.

24. The method of claim 20, further comprising, prior to step b) isolating nuclei of the tumor cells from said solid tumor.

25. The method of claim 24, further comprising, prior to step b) separating tumor nuclei from non-tumor nuclei.

26. The method of claim 23, wherein said isolating comprises single parameter or muliparameter flow sorting.

27. The method of claim 23, wherein said isolating comprises DNA content based flow sorting.

28. The method of claim 20, wherein said subjecting to conditions of step c) comprises exposing said nucleic acid to a copy number array.

29. The method of claim 20, wherein said at least one nucleic acid damaging agent is an alkylating agent.

30. The method of claim 29, wherein said alkylating agent is a metal salt.

31. The method of claim 30, wherein said metal salt is selected from the group consisting of Carboplatin, Cisplatin, and Oxaliplatin.

32. The method of claim 20, wherein said at least one nucleic acid repair inhibitor is a polymerase inhibitor.

33. The method of claim 32, wherein said polymerase inhibitor is an inhibitor of poly ADP ribose polymerase (PARP).

34. The method of claim 33, wherein said inhibitor is Olaparib.

35. The method of claim 20, wherein said solid tumor is a pancreatic tumor.

36. The method of claim 35, wherein said solid tumor is pancreatic ductal adenocarcinoma (PDA).

37. The method of claim 20, wherein said solid tumor is a cancer of the brain, ovary, breast, or colon.
